(19) United States
(12) Patent Application Publication
(10) Pub. No.: US 2016/0239732 A1
(43) Pub. Date: Aug. 18, 2016

(54) SYSTEM AND METHOD FOR USING NUCLEIC ACID BARCODES TO MONITOR BIOLOGICAL, CHEMICAL, AND BIOCHEMICAL MATERIALS AND PROCESSES

(71) Applicant: Clear Labs Inc., Menlo Park, CA (US)

(72) Inventor: Sasan Amini, Redwood City, CA (US)

(73) Assignee: CLEAR LABS INC., Menlo Park, CA (US)

(21) Appl. No.: 14/947,882

(22) Filed: Nov. 20, 2015

Related U.S. Application Data
(60) Provisional application No. 62/082,610, filed on Nov. 20, 2014.

(51) Int. Cl. G06K 19/02; C12Q 1/68

(52) U.S. Cl. CPC G06K 19/022; C12Q 1/6874; G06K 2019/06234

(57) ABSTRACT

Aspects of the embodiments use nucleic acid sequence (NS) based barcodes (NS tracking barcodes) in different capacities to track, trace, monitor, optimize, and troubleshoot complex biological, chemical, and biochemical processes. At least two forms of the NS tracking barcodes are described herein: ported NS tracking barcodes and process NS tracking barcodes. Ported NS tracking barcodes are designed such that they will not be modified during the process, and their sequence can indicate time or location of manufacture information, as well as an indication of success of a DNA processing step. Process NS tracking barcodes can be more complicated than the ported NS tracking barcodes, as they can be modified during the course of DNA processing, so that they can provide specific information regarding whether a desired nucleic acid process or reaction worked or did not. Process NS tracking barcodes can be synthesized such that they can used as a substrate for the reaction and get modified. Many nucleic acid sequencing or other molecular counting techniques are used to quantify the modified and unmodified NS tracking barcodes to calculate conversion efficiency, and correct for amplification bias or inefficiencies, as well as identifying other processing issues. The nucleic acids used in NS tracking barcodes have robust structures, dense information content, and can be readily synthesized and sequenced.

### TECHNICAL FIELD

The embodiments described herein relate generally to tracking of materials, and more specifically to systems, methods, and modes for the use of nucleic acids in tracking biological, chemical, and biochemical materials and processes.

### SEQUENCE LISTING

The sequence listing is described as follows and incorporated by reference in its entirety. The length of the nucleotide acid sequence bar code, as defined hereinbelow, is a flexible region and is dictated by the desired complexity of the barcode space. For any nucleotide position dedicated to the barcode region, at least 4 possible options become available to increase barcode diversity, meaning A, T, C, G for DNA based barcodes; A, U, G, C for RNA based barcodes; with methylation, inclusion of artificial nucleotides or other modification being able to increase the number of possible options. To be more specific, a DNA-barcode of length N with 4 possible options at each position can have an overall complexity of 4N. In designing the barcode space, it is experimentally desirable to have all possible barcodes have Hamming distance values larger than 1. The Hamming distance between two strings of equal length is the number of positions at which the corresponding symbols are different. Two different barcodes with Hamming distance of 1 are more likely to be cross-assigned to each other by mistake. For examples, if the total length of the barcode region is 6, two sequences of ATGGTC and ATGCTC are not ideal choices for the barcode since their Hamming distance is only 1. In the exemplary embodiment, the nucleotide bases are designated in such sequence listing according to the WIPO Standard ST.25 (1998), as follows: r=g or a (purine); y=tlu or c (pyrimidine); m=a or c; (amino); k=g or tlu (keto); s=g or c (strong interactions 3 H-bonds); w=a or tlu (weak interactions 2H-bonds); b=g or cor tlu (not a); d=a or g or tlu (not c); h=a or cor tlu (not g); v=a or g or c (not t, not u); and n=a or g or cor tlu (unknown, or other; any.)

### BACKGROUND

DNA sequencing refers to determining the order of nucleotide building block in the structure of an oligonucleotide or polynucleotides, and gene (DNA) sequencing refers to the same activity for a gene. Early sequencing methods were very time consuming. In 1975, Frederick Sanger invented the technique that bears his name, where fluorescent-labeled DNA fragments are separated according to their lengths on a polyacrilimide gel, and the base at the end of each fragment is identified by the dye with which it reacts. The technique was intensive of both time and labor due to the nature of gel preparation and running, and the large sample sizes needed. Sanger then invented “shotgun” sequencing, where random pieces of DNA are isolated from the host genome to be used as primers for the PCR amplification of the entire genome. Here, amplified DNA portions are assembled by the overlapping regions in order to form contiguous transcripts known as contigs, and then custom primers are used to elucidate gaps between these contigs to sequence the genome.

Many generations of sequencing succeeded these early techniques. In the 1980's, Pohl developed a non-radioactive method for transferring DNA molecules of sequencing reaction mixtures onto an immobilizing matrix during electrophoresis; GATC Biotech created a commercial DNA sequencer for sequencing the yeast Saccharomyces cerevisiae chromosome II; Hood's lab at the Caltech created a semi-automated DNA sequencing methodology; and Applied Biosystems sold the first completely automated sequencing machine, inter alia.

The 1990's began with the National Institute of Health's commencement of large-scale sequencing trials (e.g., for Mycoplasma capricolum, Escherichia coli, Caenorhabditis elegans, and Saccharomyces cerevisiae). Ventor sequenced human cDNA sequences to capture the coding fraction for the human genome. The Institute for Genomic Research published the first complete genome of the bacterium Haemophilus influenzae. The 90's were also marked by early next-generation sequencing techniques, including the pyrosequencing work of Pål Nyrén and Mostafa Ronaghi at the Royal Institute of Technology in Stockholm.

Next-generation methodologies have been extended through today's methods for resequencing, epigenome characterization, transcriptome profiling (RNA sequencing), and DNA-protein interactions (ChIP-sequencing) and the like. Many advanced techniques have in fact been developed over the past two decades, as sequencing has gone from the basic research stage to implementation and production on a massive scale for the betterment of biological science in general and its beneficial applications specifically. For example, in relation to just the transcriptome, next-generation techniques are offering new capabilities in co-transcriptional modification, mutations, alternative splicing, gene fusion and changes in gene expression. Today's techniques must be process and efficiency oriented, as high-throughput and ultra-high throughput sequencing parallelize the sequencing process, producing thousands or millions of sequences concurrently.

However, while high-throughput systems increase speed and power, there is tremendous room for improvement in the area of efficiency. What is needed is qualitative and/or quantitative information about the processing in order to improve it. This is true not only for modern sequencing techniques specifically, but in general to all next-generation biological, chemical and biochemical processing techniques. What is needed is a system, and corresponding method, for tracking, tracing, monitoring, optimizing and troubleshooting complex processes, and in particular for the fast-paced world of modern genomic sequencing. The proposed solution has to be cost-effective and at the same time flexible, so it can be adapted and applied to different applications.

### SUMMARY

An object of the embodiments is to substantially solve at least the problems and/or disadvantages discussed above, and to provide at least one or more of the advantages described below.

It is therefore a general aspect of the embodiments to provide a system and method for using nucleic acid barcodes that will obviate or minimize problems of the type previously described.

According to aspects of the embodiments, nucleic acid sequences (NS), such as for example oligonucleotides and/or polynucleotides, which can be tags, barcodes, indices, molecular identifiers, or other tracking methods/systems, and as otherwise used and/or defined herein (referenced herein as “barcodes,” “NS” and by other identification means) can be used in numerous capacities to track, trace, monitor, optimize, and troubleshoot complex biological, chemical, and biochemical processes. The embodiments are not limited to the use of nucleic acid sequences, as for example any type of biologically based methods and systems for tagging and the aforementioned can be used.

At least two forms of the NS tracking barcodes are described herein: ported NS tracking barcodes and process NS tracking barcodes. Ported NS tracking barcodes are designed such that they will not be modified during the process, and their sequence can indicate time or location of manufacture information, as well as an indication of success of a nucleic acid (for example, DNA) processing step. Process NS tracking barcodes are more complicated than the ported NS tracking barcodes, as they can be modified during the course of a nucleic acid (for example, DNA) processing, so that they can provide specific information whether a desired nucleic acid process or reaction worked or not. Process NS tracking barcodes can be synthesized such that they can used as a substrate for the reaction and get modified. It is noteworthy that reference is made to DNA herein, but the embodiments are applicable to any type of nucleic acid sequence. Many conventional nucleic acid sequencing or other molecular counting techniques can be used to quantify the modified and unmodified NS tracking barcodes to calculate conversion efficiency, and correct for amplification bias or inefficiencies, as well as identifying other processing issues. The nucleic acids used in NS tracking barcodes have robust structures, dense information content, and can be readily synthesized and sequenced.

Additionally, certain embodiments of the present invention are set forth in Exhibit A hereto.

### BRIEF DESCRIPTION OF THE DRAWINGS

The above and other objects and features of the embodiments will become apparent and more readily appreciated from the following description of the embodiments with reference to the following Figures, wherein like reference numerals refer to like parts throughout the various Figures unless otherwise specified, and wherein:

FIG. 1 illustrates a flow chart of a method for using nucleic acids to track chemical, biological, and biochemical processes according to certain embodiments.

FIG. 2 illustrates a block diagram of an arrangement of a plurality of nucleic acid tracking barcodes with one or more experiment or sample specific deoxyribonucleic acid (DNA) molecules according to certain embodiments.

FIGS. 3A and 3B illustrate block diagrams of an arrangement of a plurality of process nucleic acid tracking barcodes with one or more sample specific DNA molecules in an unsuccessful and successful polymerase chain reaction (PCR) process, respectively, wherein process nucleic acid tracking barcodes are used to show the success or not of the PCR reaction process according to certain embodiments.

FIG. 4 illustrates block diagrams of an arrangement of a plurality of process nucleic acid tracking barcodes with a sample specific DNA molecule in an unsuccessful and successful restriction digestion process, wherein the process nucleic acid tracking barcodes are used to show the success or not of the restriction digestion process according to certain embodiments.

FIG. 5 illustrates block diagrams of an arrangement of a plurality of process nucleic acid tracking barcodes with sample specific DNA molecules in targeted bisulfite sequencing and methylation process, wherein the process nucleic acid tracking barcodes are used to show the results of the targeted bisulfite sequencing and methylation process according to certain embodiments.

FIGS. 6A, 6B, and 6C illustrate block diagrams of an arrangement of a plurality of ported nucleic acid tracking barcodes with sample specific DNA molecules in a process for evaluating DNA extraction from a sample using tissue lysis and DNA purification techniques, wherein the ported nucleic acid tracking barcodes are used to show the results of the tissue lysis and DNA purification processes according to certain embodiments.

FIG. 7 illustrates block diagrams of an arrangement of a plurality of ported and process nucleic acid tracking barcodes with two separate sample specific DNA molecules used in a plurality of processes for subsequent identification of the two entities that provided the two separate sample specific DNA molecules, wherein the process and ported nucleic acid tracking barcodes can be used to evaluate separately each of the process steps used to identify the two entities according to certain embodiments.

### DETAILED DESCRIPTION

The embodiments are described more fully hereinafter with reference to the accompanying drawings, in which embodiments of the inventive concepts are shown. In the drawings, the size and relative sizes of layers and regions may be exaggerated for clarity. Like numbers refer to like elements throughout. In reference to the figures comprising the drawings, and referenced in the Brief Description of the Drawings, the following is a list of the elements of in numerical order:
100 Method For Using Nucleic Acids to Track Chemical, Biological, and Biochemical processes;
102-112 Steps for Method 100;
200 Block Diagram of an Arrangement of a Plurality of Nucleic Acid Tracking Barcodes with Test/Sample DNA Molecules;
202 Ported Nucleic Acid Tracking Barcodes;
204 Process Nucleic Acid Tracking Barcodes;
206 First Sample DNA Molecules;
300 First DNA Process use of Nucleic Acid Tracking Barcodes as Process Barcodes;
400 Second DNA Process use of Nucleic Acid Tracking Barcodes as Process Barcodes;
402 Second Sample DNA Molecules;
500 Third DNA Process use of Nucleic Acid Tracking Barcodes as Process Barcodes;
502 Third Sample DNA Molecules;
600 First DNA Process use of Nucleic Acid Tracking Barcodes as Ported Barcodes;
602 Fourth Sample DNA Molecules;
604 Second Part of Nucleic Acid Tracking Barcode;
700 DNA Process use of Nucleic Acid Tracking Barcodes as Both Process and Ported Barcodes;
702 First Type of DNA Sample Molecule;
704 Second Type of DNA Sample Molecule;
706 Mixed DNA Sample; and
708 DNA Process Mixture.

Regardless of the foregoing drawings, Exhibit A, and the accompanying description, the embodiments can be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

In certain embodiments, for simplicity, nucleotide sequences (NS), barcodes and other terms (as defined below) are discussed with regard to the terminology and structure of a systems and methods for determining the presence, or lack thereof, of DNA in biological or chemical samples. However, these embodiments are not limited to these systems but can be applied to other systems and methods for determining the presence or absence of specific nucleic acid materials, as well as determining the efficacy of, and tracking of materials used in, nucleic acid processes.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” on “in an embodiment” in various places throughout the specification is not necessarily referring to the same embodiment. Further, the particular feature, structures, or characteristics can be combined in any suitable manner in one or more embodiments.

Aspects of the embodiments use NS based barcodes (as defined hereinbelow) in different capacities to track, trace, monitor, optimize, and troubleshoot complex biological, chemical, and biochemical processes. At least two forms of the NS tracking barcodes are described herein: ported NS tracking barcodes and process NS tracking barcodes. Ported NS tracking barcodes are designed such that they will not be modified during the process, and their sequence can indicate time or location of manufacture information, as well as an indication of success of a DNA processing step. Process NS tracking barcodes are more complicated than the ported NS tracking barcodes, as they can be modified during the course of DNA processing, so that they can provide specific information whether a desired nucleic acid process or reaction worked or not. Process NS tracking barcodes can be synthesized such that they can used as a substrate for the reaction and get modified. Many conventional nucleic acid sequencing or other molecular counting techniques can be used to quantify the modified and unmodified NS tracking barcodes to calculate conversion efficiency, and correct for amplification bias or inefficiencies, as well as identifying other processing issues. The nucleic acids used in NS tracking barcodes have robust structures, dense information content, and can be readily synthesized and sequenced.

Used throughout the specification are several acronyms, the meanings of which are provided as follows: DNA: deoxyribonucleic acid; RNA: ribonucleic acid; FRET: fluorescence resonance energy transfer; NS: nucleic acid sequence; PCR: and polymerase chain reaction.

As used herein and understood by skilled persons, nucleic acids are any of the group of complex compounds consisting of linear chains of monomeric nucleotides whereby each monomeric unit is composed of phosphoric acid, sugar and nitrogenous base, and involved in the preservation, replication, and expression of hereditary information in every living cell. Nucleic acids may be, for example, in the form of deoxyribonucleic acid (DNA) or ribonucleic acid (RNA) molecules containing the genetic information important for all cellular functions and heredity.

As used herein and also understood by skilled persons, DNA itself is a double-stranded nucleic acid that contains the genetic information for cell growth, division, and function, and is composed of two strands that twist together to form a helix, with each strand consisting of alternating phosphate and pentose sugar (2-deoxyribose), and attached on the sugar is a nitrogenous base, which can be adenine (A), thymine (T), guanine (G), or cytosine (C), with the bases A and T pairing, and G and C pairing. Again, as well known and understood, RNA itself is a nucleic acid that is generally single stranded, and double stranded in some viruses, and plays a role in transferring information from DNA to protein-forming system of a cell, with a molecule consisting of a long linear chain of nucleotides, with each nucleotide unit comprising a sugar, phosphate group and a nitrogenous base, and differing from a DNA molecule in that the sugar backbone is a ribose (versus deoxyribose in DNA), and the bases comprise A, G, C and uracil (U) (versus thymine in DNA). For most organisms, RNAs are involved in: post-transcriptional modification or DNA replication (such as snRNA, snoRNA and others), protein synthesis (such as mRNA, tRNA, rRNA, and others) and gene regulation (such as miRNA, siRNA, tasiRNA, and others).

As used herein and also understood by skilled persons, the foregoing A, C, G, T and U are nucleotides, composed of a nucleobase (which may also be referenced as a nitrogenous base), a five-carbon sugar, which is either ribose or 2-deoxyribose, and one or more (depending on the definition) phosphate groups. Authoritative chemistry sources typically state that a nucleotide refers only to a molecule containing one phosphate, while common usage with skilled persons may extends the definition to include molecules with two or three phosphate groups. Accordingly, as used herein, the term nucleotide refers to a nucleoside monophosphate, a nucleoside diphosphate or nucleoside triphosphate, as well as any other variations that may be used by skilled persons.

As used herein and also understood by skilled persons, linear sequence of up to 20 nucleotides joined by the phosphodiester bonds are commonly termed oligonucleotides, and above this length, are commonly termed polynucleotides. The inventive embodiments are applicable to oligonucleotides, polynucleotides or any other nucleotide sequences (NS). NS as referenced herein may be used to track chemical, biological, and biochemical processes, and any other more general or more specific applicable uses, including without limitation DNA sequencing, library construction, DNA microarrays, artificial gene synthesis, ASO analysis, fluorescent in situ hybridization, antisense therapy, polymerase chain reaction (PCR), molecular probes, among others.

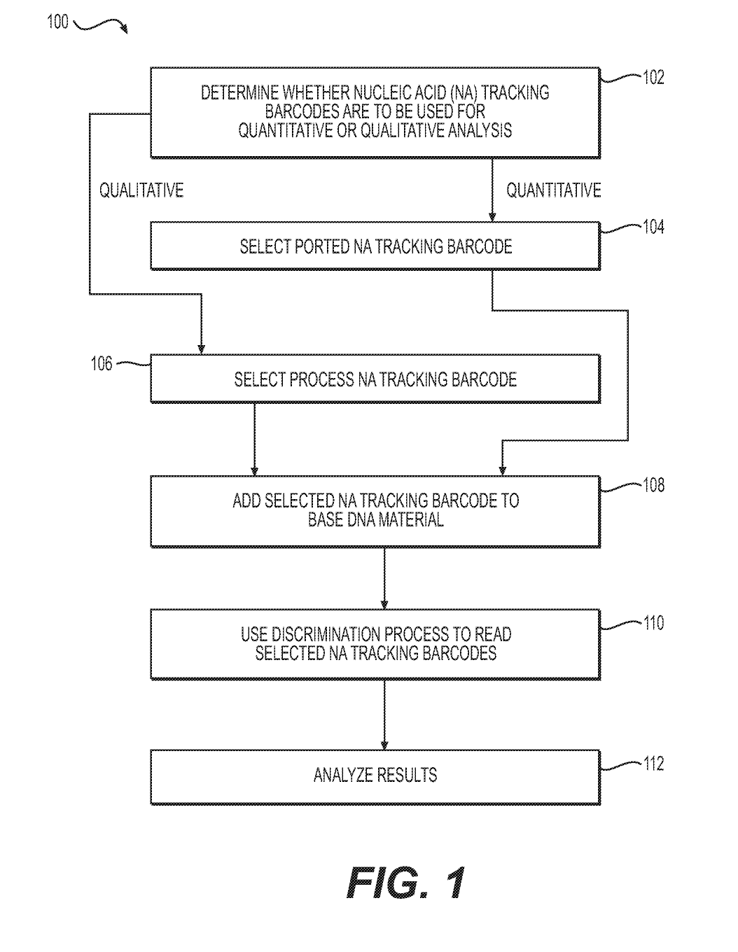

FIG. 1 illustrates a flow chart of method 100 for using NS to track chemical, biological, and biochemical processes according to an embodiment. According to aspects of the embodiments, NS are used as tools for traceability and tracking simple or multi-step procedures, and are referenced as tracking barcodes or NS tracking barcodes. In certain embodiments, there are two types of NS tracking barcodes as discussed below.

According to aspects of the embodiments, NS tracking barcodes with known sequences or structural configurations (i.e., the arrangement of the nucleotides “A,” “C,” “T,” or “G”) can be used to provide information about time and date to keep track of the time the barcode oligos were synthesized, or the time DNA samples are analyzed.

According to further aspects of the embodiment, NS tracking barcodes can be used to convey location information, e.g. where samples are coming from. They could also contain information about discrete parts of a multi-step process. As those of skill in the art can appreciate, arrangements of the four nucleotides, in different lengths, can be used as a code, no different from a binary code, or hexadecimal code, or any other of a plurality of codes, that can be arranged in certain known manners (such as a bar code) to convey time, date, serial or batch number, and the like.

NS tracking barcodes, as stable nucleic acids, can be added to a physical compartment or tube used in the procedure, or to certain components of the process, and can be collected at the end point of the process. Following collection, the entire sample of DNA molecules can be detected or sequenced with substantially any nucleic acid detection or sequencing technology. According to aspects of the embodiments, the use of NS tracking barcodes can provide both qualitative and quantitative information regarding the whole process, or certain components of it, based on the design, as described in greater detail below. According to further aspects of the embodiments described herein, NS tracking barcodes can be used in a new capacity as a multi-functional tracking system that can provide both quantitative and qualitative data about different components of a multi-step procedure or an entire system.

The use of DNA as a tracking barcode has several advantages. DNA has a robust structure, which makes it likely to withstand the different processes that the other DNA sample molecules will have to endure. This robust structure provides a redundancy in copy number that can be used to correct for any context or processing error. Further, even a small amount of DNA, such as is used to make the NS tracking barcodes, has a dense amount of information content. This is shown in the number of different coding schemes that can be developed based on the arrangements of the nucleotides. Further still, with the dropping cost of DNA synthesis and DNA sequencing, the use of DNA for NS tracking barcodes provides a very powerful, scalable, but at the same time cost-effective platform for process engineering, trouble-shooting, and optimization.

According to aspects of the embodiment, NS tracking barcodes can be designed either as process NS tracking barcodes or ported NS tracking barcodes. According to an aspect of the embodiments, ported NS tracking barcodes do not get modified during the process and their presence or absence provides information about the success of the procedure. According to a further aspect of the embodiments, process NS tracking barcodes can be used as a substrate for the reaction and do get modified. Furthermore, nucleic acid sequencing or other molecular counting techniques can be used to quantify the modified and unmodified NS tracking barcodes, therefore calculating conversion efficiency. In case of DNA amplification processes, NS tracking barcodes can be used to correct for any amplification bias or inefficiencies, providing more quantitative results.

According to aspects of the embodiments, NS tracking barcodes are fragments of nucleic acids with defined sequences and/or chemical structures. The chemical structures of the NS tracking barcodes can be naturally occurring versions of DNA or ribonucleic acid (RNA; i.e., the NS tracking barcodes can be made from deoxyribonucleotide and/or ribonucleotide building blocks), or artificially modified ones (e.g. locked nucleic acids (LNA), bridged nucleic acids (BNA)), among others.

As those of skill in the art can appreciate, LNAs are a class of high-affinity RNA analogs in which the ribose ring is “locked” in the ideal conformation for Watson-Crick binding. As a result, LNA oligonucleotides exhibit thermal stability when hybridized to a complementary DNA or RNA strand. BNAs, however, are based on multi-functional synthetic RNA analogues that can be used in place of the first generation bridged nucleic acids known as LNAs. According to further aspects of the embodiments, NS tracking barcodes can further be designed based on a pre-defined (pre-designed) set of sequences or they could be random sets of sequences.

According to an aspect of the embodiments, NS tracking barcodes can be synthesized (or made) with enzymatic reactions. These enzymatic reactions can use ribonucleic acids (RNA) and/or DNA polymerases. According to further aspects of the embodiments, the enzymatic reactions that can be used to synthesize NS tracking barcodes can further include a chemical synthesis scheme, which, for example, uses phosphoramidite, which can be synthesized on a solid state (i.e. beads, or on a chip), in solution, in a compartmentalized fashion, or in bulk (i.e., with split-pool synthesis), among other known processes.

As those of skill in the art can appreciate, RNA, which stands for ribonucleic acid, is a polymeric molecule made up of one or more nucleotides. A strand of RNA can be thought of as a chain with a nucleotide at each chain link. Each nucleotide is made up of a base (adenine, cytosine, guanine, and uracil, typically abbreviated as A, C, G and U), a ribose sugar, and a phosphate. The structure of RNA nucleotides is very similar to that of DNA nucleotides, with the main difference being that the ribose sugar backbone in RNA has a hydroxyl (—OH) group that DNA does not. This gives DNA its name: DNA stands for deoxyribonucleic acid. Another minor difference is that DNA uses the base thymine (T) in place of uracil (U).

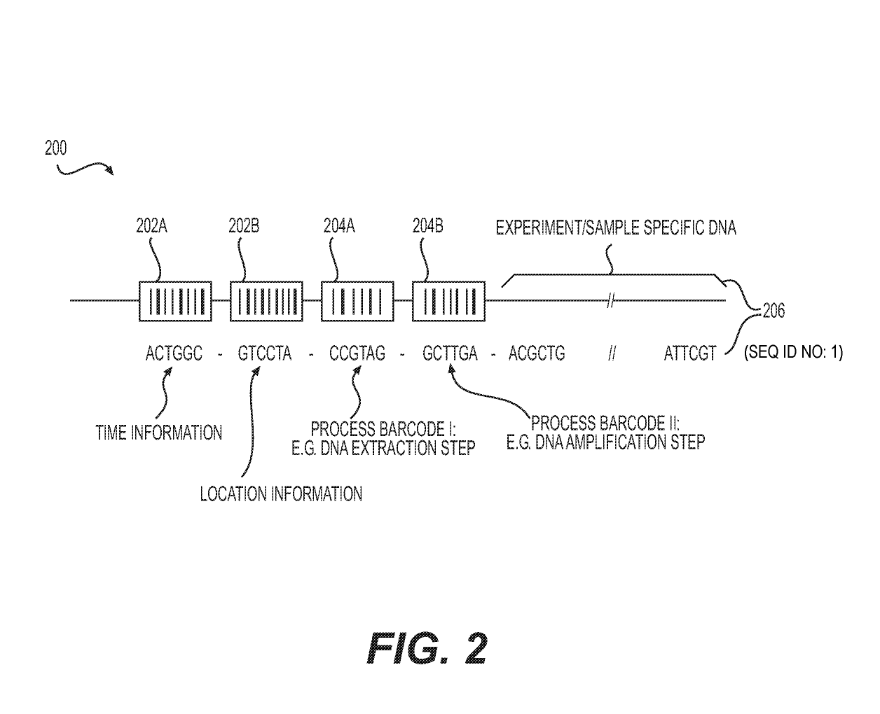

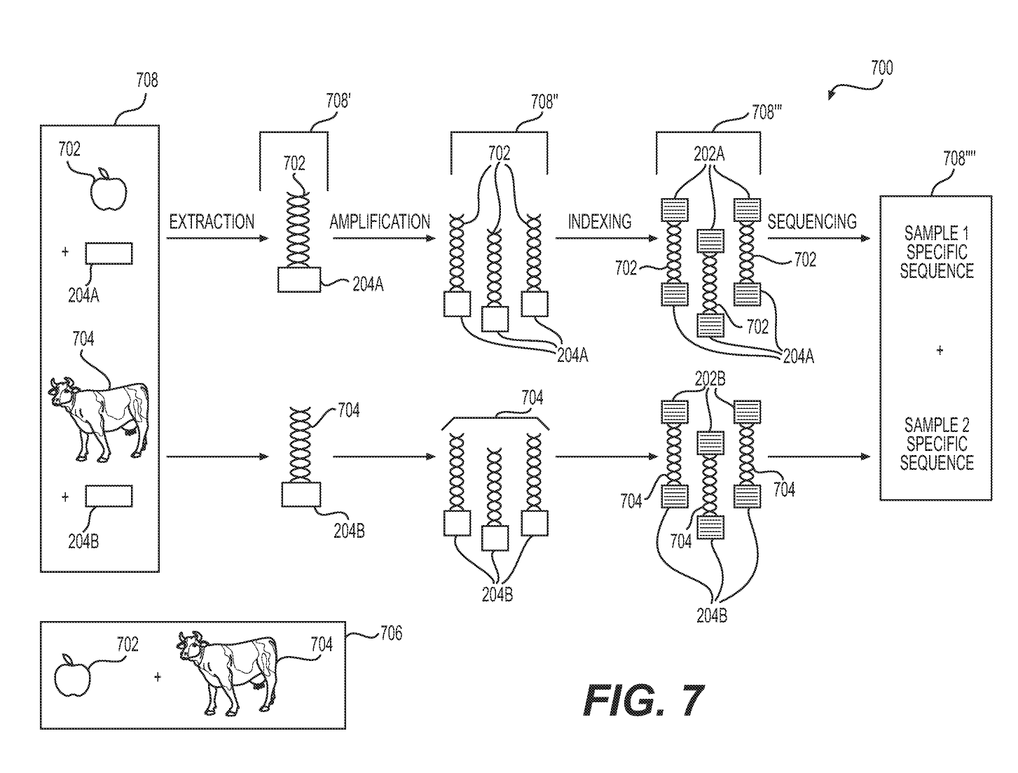

Attention is now directed to FIG. 1, which illustrates a flow chart of method 100 for using NS barcodes to track chemical, biological, and biochemical processes according to an embodiment, and FIG. 2 illustrates block diagram 200 of an arrangement of a plurality of ported NS tracking barcodes 202 and process NS tracking barcodes 204 with one or more experiment or sample specific deoxyribonucleic acid (DNA) molecules 206 according to an embodiment. Method 100 begins with the determination of whether to use ported NS tracking barcodes 202, or process NS tracking barcodes 204. As discussed in regard to FIG. 7, there is shown and discussed an example of at least one process that includes both ported and process NS tracking barcodes 202, 204. However, as those of skill in the art can appreciate, in fulfillment of the dual purposes of clarity and brevity, the principle discussion will be in regard to a choice between one or the other of ported and process NS tracking barcodes 202, 204, but such discussion should not be taken in a limiting manner.

As briefly discussed above, NS tracking barcodes can be categorized into two different types: ported NS tracking barcodes 202, and process NS tracking barcodes 204. FIG. 2 illustrates first sample DNA molecule 206 with both ported NS tracking barcodes 202 and process NS tracking barcodes 206. In FIG. 2, ported NS tracking barcodes 202a can be used to indicate a time of “manufacture” of first sample DNA molecule 206, and ported NS tracking barcodes 202b can be used to indicate a place or location of manufacture of first sample DNA molecule 206. The time/place distinction can be discerned by the pattern of nucleotides (A, C, T, and G) within ported NS tracking barcodes 202a,b. In a similar manner, because process NS tracking barcodes 204a,b can (but not necessarily) change as a result of processing undergone by first sample DNA molecule 206, they can indicate that a DNA extraction step has taken place, and that a DNA amplification step has taken place. According to further aspects of the embodiments, process NS tracking barcodes 204 do not necessarily have to change during any of the DNA processing steps.

If the use of method 100 determines that ported NS tracking barcodes 202 should be used (quantitative analysis (i.e., how much of something occurred)), method 100 proceeds to step 104 (“Quantitative” path from decision step 102), wherein a ported NS tracking barcode 202 is selected. Or, if the user determines that a process NS tracking barcode 204 should be used (qualitative analysis (i.e., how well did the process work), method woo proceeds to step 106 (“Qualitative” path from decision step 102), wherein a process NS tracking barcode 204 is selected. According to further aspects of the embodiments, one major difference between ported and process NS tracking barcodes 202, 204 is based on their structure.

As described above, NS tracking barcodes can be split into separate modules: ported NS tracking barcodes 202, and process NS tracking barcodes 204. Ported NS tracking barcodes 202 can contain time and date information to keep track of the time the ported NS tracking barcodes 202 were synthesized, or the time sample DNA molecules were analyzed. Ported NS tracking barcodes 202 can contain location information, such that a place of “manufacture” or origin of the sample DNA molecules. NS tracking barcodes can also contain information about discrete parts of a multi-step process; as described above, these are referred to as process NS tracking barcodes 204. As shown in FIG. 2, first sample DNA molecules 206 can vary from process to process, and it generally a piece of DNA with biological information that is intended to be sequence.

According to further aspects of the embodiments, process NS tracking barcodes 204 are modified during the course of the processing the sample DNA molecules undergo, so they can provide specific information whether a desired nucleic acid process or reaction worked or not. As those of skill in the art can appreciate, DNA molecule processes can include PCR, nucleic acid methylation, bisulfite treatment, restriction enzyme cleavage, and whole genome amplification, among others. As those of skill in the art can appreciate, PCR is a biomedical technology in molecular biology used to amplify a single copy or a few copies of a piece of DNA across several orders of magnitude, generating thousands to millions of copies of a particular DNA sequence.

Process NS tracking barcodes 204 can be synthesized in a such a way that it can used as a substrate for the reaction and get modified.

Furthermore, according to still further aspects of the embodiments, nucleic acid sequencing or other molecular counting techniques can be used to quantify the modified and unmodified process NS tracking barcodes 204 such that conversion efficiency can be calculated.

In case of DNA amplification processes, process NS tracking barcodes 204 can be used to correct for any amplification bias or inefficiencies, providing more quantitative results, in addition to the qualitative information.

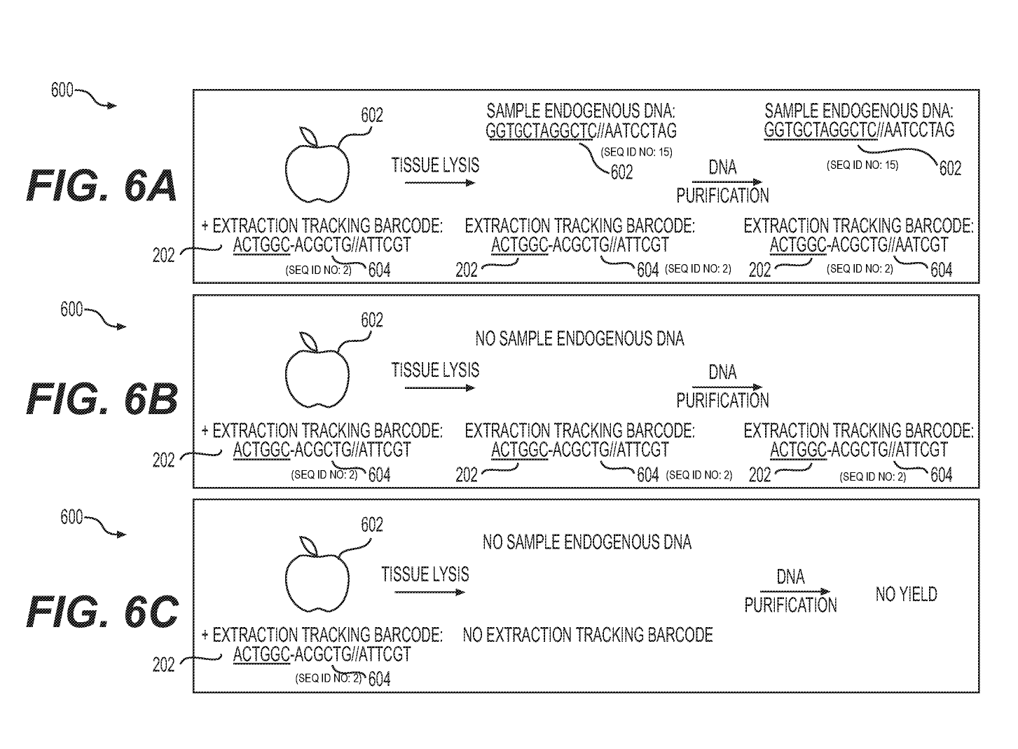

Ported NS tracking barcodes 202 are simpler versions of process NS tracking barcodes 204 in that they will not be modified during the process, and their sequence can include time or location information, encoded in the particular sequence of nucleotides, as discussed above. According to further aspects of the embodiments, the presence of ported NS tracking barcodes 202 can indicate the success of a step. By way of non-limiting example, ported NS tracking barcodes 202 can be added to raw materials of a process, e.g., lysis buffer of a DNA extraction process, and if they are found in the elution step, it indicates that the purification step has worked. This example process is discussed below in regard to FIG. 6.

Referring back again to FIG. 1, method 100 proceeds to step 108 following either of steps 104 and 106. In method step 108, the selected NS tracking barcode (either of ported NS tracking barcode 202 or process NS tracking barcode 204 (and in some cases, both) is added to the base DNA material. Following method step 108, method 100 uses a properly selected discrimination process in method step 110 to determine if, and how much of ported NS tracking barcode 202 or process NS tracking barcode 204 is present in the processed DNA material.

According to aspects of the embodiments, both of ported and process NS tracking barcode 202, 204 can be discriminated with substantially any nucleic acid detection method, including sequencing, hybridization, blotting, optical reading of fluorescence labels and fluorescence resonance energy transfer (FRET), among other methods. According to further aspects of the embodiments, any discrimination process that is used should have the resolution to discriminate all different barcodes that are likely to be present in any process. According to still further aspects of the embodiments, the selected discrimination process can provide a unique read-out of the NS tracking barcodes such that enough information is available to uniquely identify the NS tracking barcodes 202, 204. The net result of the discrimination step is to provide a count of the different sample DNA molecules, including the NS tracking barcodes, and whether and to what extend process NS tracking barcode 204 may have changed. Such results can then be used in method step 112, wherein method 100 analyzes the results of the discrimination process to ascertain the qualitative or quantitative results.

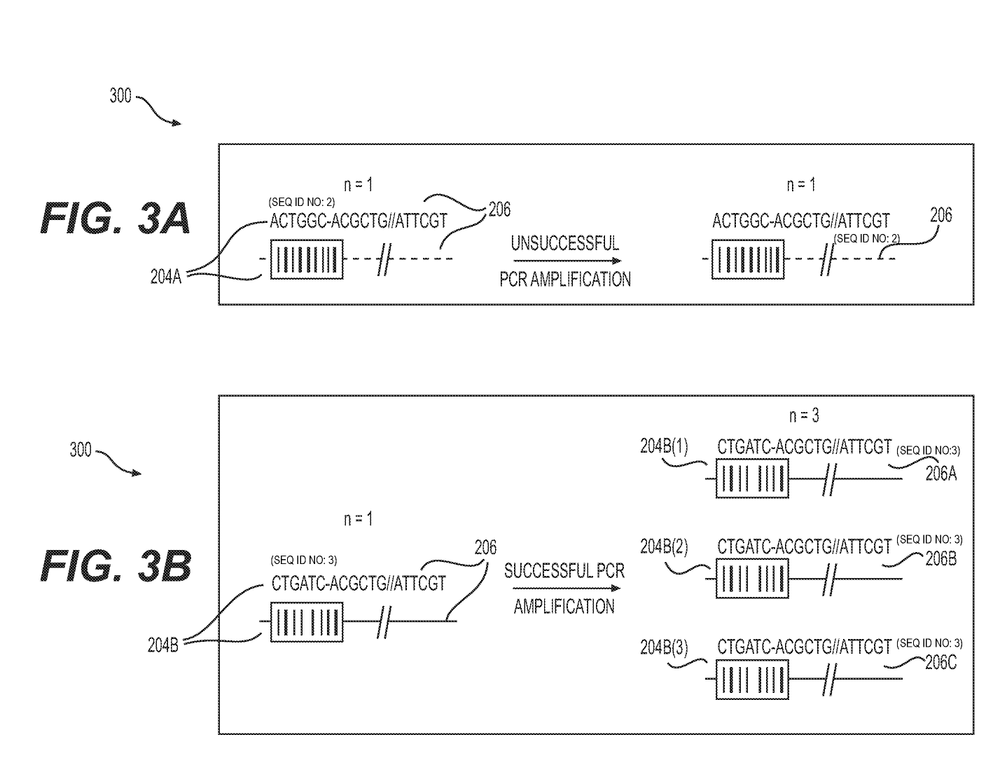

Attention shall now be directed to FIGS. 3, 4, 5, 6, and 7, wherein specific examples of method 100 will be discussed in regard to use of process NS tracking barcodes 204, ported NS tracking barcodes 202, and one example in which both ported and process NS tracking barcodes 202, 204 can be used.

FIGS. 3A and 3B illustrate block diagrams of an arrangement of a plurality of process NS tracking barcodes 204 with one or more sample specific DNA molecules 206 in an unsuccessful and successful polymerase chain reaction (PCR) process, respectively, wherein process NS tracking barcodes 204 are used to show the success or not of the PCR reaction process according to an embodiment. FIGS. 3A and 3B illustrate first DNA process 300 that uses process NS tracking barcodes 204 according to an embodiment.

FIG. 3 illustrates an example of use of process NS tracking barcodes 204 for evaluating amplification efficiency of a PCR reaction. Process NS tracking barcodes 204 are schematically shown as the first 6 nucleotides [ACTGGC] on the left side of first sample DNA molecule 206 (i.e. 5′ end of the molecule; as those of skill in the art can appreciate, the asymmetric ends of DNA strands are called the 5′ (five prime) and the 3′ (three prime) ends, with the 5′ end having a terminal phosphate group and the 3′ end a terminal hydroxyl group). First sample DNA molecule 206 is the same (i.e. ACGCTG//ATTCGT) in both FIGS. 3A, and 3B. In FIG. 3A, an unsuccessful PCR reaction has occurred in which the copy number of the barcode does not increase (i.e., n=1 before and after the unsuccessful PCR amplification reaction), while FIG. 3B illustrates a successful example in which the copy number increases (n equals 1 before and n equals 3 after successful PCR amplification (i.e., 206a, 206b, 206c)). According to further aspects of the embodiment, even if the components of FIGS. 3A and 3B are mixed with each other, the tracking barcodes allow identification of the successful component.

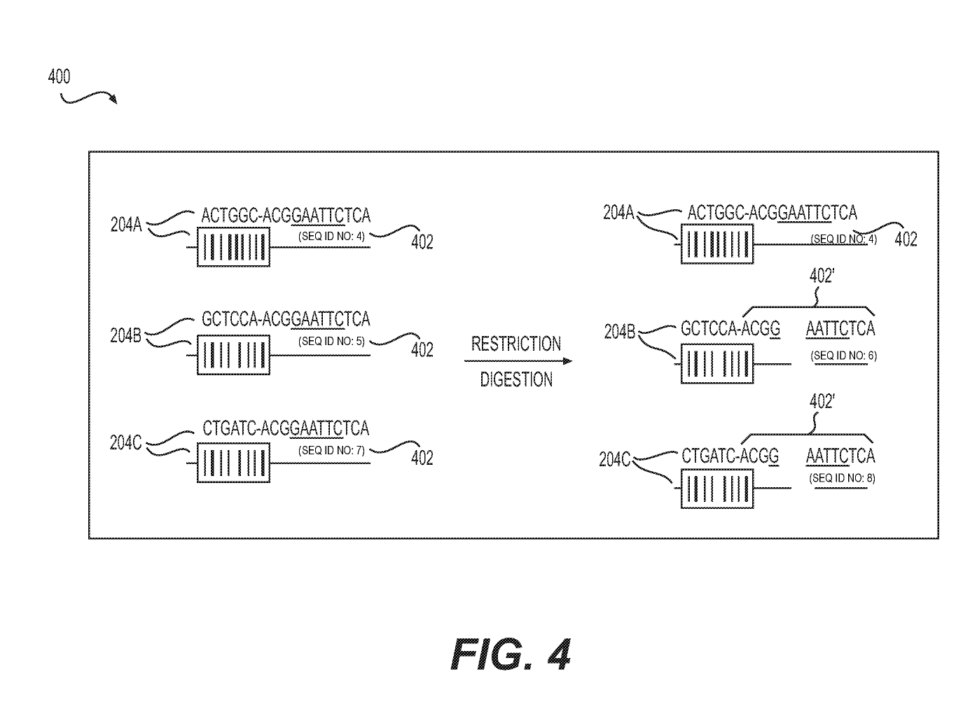

FIG. 4 illustrates block diagrams of an arrangement of a plurality of process NS tracking barcodes 204 with second sample specific DNA molecule 402 in an unsuccessful and successful restriction digestion process, wherein the process NS tracking barcodes 204 are used to show the success or not of the restriction digestion process according to an embodiment. FIG. 4 illustrates second DNA process 400 that uses process NS tracking barcodes 204 according to an embodiment.

In FIG. 4, according to an embodiment, restriction enzyme EcoRI with the recognition sequence, GAATTC (underlined in the schematic figure below), was chosen. All process NS tracking barcodes 204a,b,c (on the left) have a unique DNA barcode sequences ACTGGC, GCTCCA, and CTGATC, respectively. Upon treatment with the enzyme, GCTCCA and CTGATC-tagged molecules (204b,c) were successfully digested while ACTGGC-tagged molecule was left intact. Using a DNA quantification method like DNA sequencing, a proportion of barcode molecules digested can be calculated and used for estimating restriction enzyme efficiency. This ratio can be estimated by a relative measure, i.e. by measuring the DNA barcode diversity of the digested library (process NS tracking barcodes 204b,c and second sample DNA barcodes 402′) versus the undigested library (process NS tracking barcodes 204a and second sample DNA barcodes 402), or by an absolute measure, i.e. counting the number of barcodes eliminated from a pool with known composition of DNA barcodes. In the specific case shown in FIG. 4, the efficiency coefficient is about 67%, because 2 second sample DNA molecules 402′ were successfully digested.

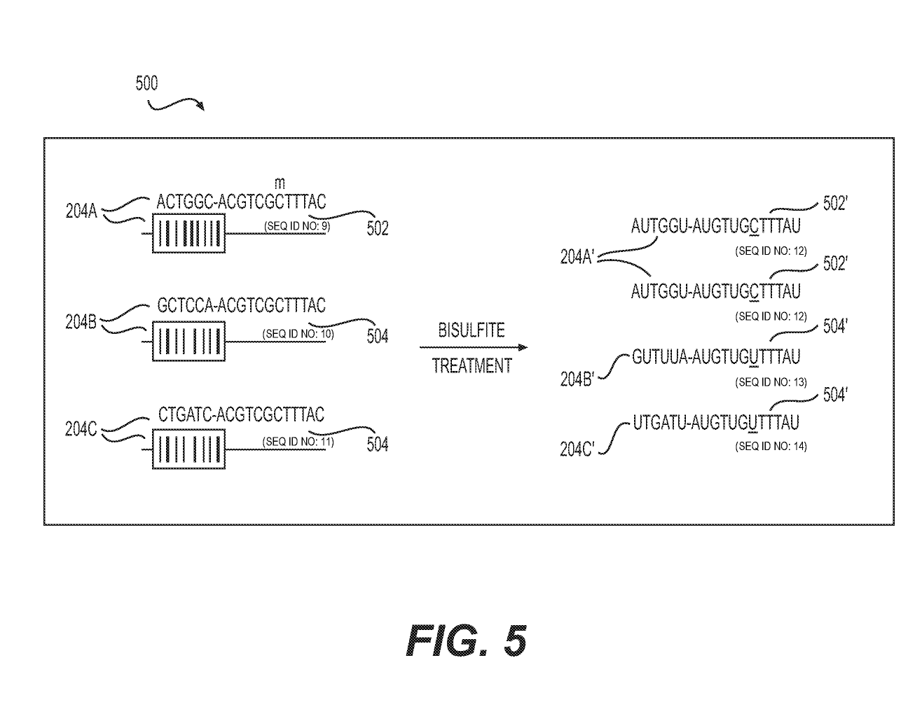

FIG. 5 illustrates block diagrams of an arrangement of a plurality of process NS tracking barcodes 204 with third sample specific DNA methylated molecules 502 and third sample specific DNA un-methylated molecules 504 in a targeted bisulfite sequencing and methylation process, wherein the process NS tracking barcodes 204 are used to show the results of the targeted bisulfite sequencing and methylation process according to an embodiment. FIG. 5 illustrates third DNA process 500 that uses process NS tracking barcodes 204 according to an embodiment.

In FIG. 5, the bisulfite treatment converts all un-methylated cytosines (those cytosine nucleotides (C) without an “m” over them, to uracil, while the methylated cytosines (underlined position) remain unchanged. All process NS tracking barcodes 204a,b,c have a unique DNA barcode sequences of ACTGGC, GCTCCA, and CTGATC, respectively. Upon the bisulfite treatment, library amplification, and preparation, sequencing can be used to determine and quantify the methylated and un-methylated residues. Process NS tracking barcodes 204 identity can be used to collapse the reads down to their clonal origin. Thus, as shown in FIG. 5, there are 4 DNA molecules on the right that can be sequenced, wherein two of them (third sample DNA methylated molecules 502′), are methylated (and two are not third sample DNA un-methylated molecules 504′), which implies that 50% of starting molecules were methylated at that cytosine residue (TCGCTT). However, among those 4, the two methylated ones (502′) share the same process NS tracking barcode 204a′, AUTGGU (following bisulfite treatment of process NS tracking barcodes 204a of ACTGGC), which means that they are amplification duplicates. Therefore, correcting for such amplification bias will yield a 1:2 ratio of methylated versus un-methylated residue which is 33%. This is useful for targeted sequencing applications where target molecules are made with an amplification step and there is a high probability of generating amplification duplicates that can skew the quantification.

FIGS. 6A, 6B, and 6C illustrate block diagrams of an arrangement of a plurality of ported NS tracking barcodes 202 with fourth sample specific DNA molecules 602 in a process for evaluating DNA extraction from a sample using tissue lysis and DNA purification techniques, wherein ported NS tracking barcodes 202 are used to show the results of the tissue lysis and DNA purification processes according to an embodiment. FIGS. 6A, 6B, and 6C illustrate first DNA process 600 that uses ported NS tracking barcodes 202 according to an embodiment.

In FIG. 6, apple tissue is shown as a representative source of fourth sample specific DNA molecules 602, but in general, samples can be of any source. Process NS tracking barcode 202 is shown as the first 6 nucleotides on the 5′ end of the complete tracking molecule, which includes second part NS tracking barcode 604. According to an embodiment, in this case, fourth sample DNA molecules 602 are not barcoded. FIG. 6A illustrates a successful reaction in which both the extraction tracking barcode (combination of ported NS tracking barcode 202 and second part NS tracking barcode 604) and fourth sample DNA molecules 602 were observed (i.e., both are present). In FIG. 6B, however, the extraction tracking barcode (202, 604) is collected, while fourth sample DNA molecules 602 is missing, which indicates a sample-specific problem. In FIG. 6C, both the extraction tracking barcode (202, 604) and fourth sample DNA molecules 602 are missing, which indicates a process-specific problem, i.e. a problem with lysis buffer spoilage and/or DNA purification columns inefficiencies. Without the tracking barcodes (202, 604), the cases shown in FIGS. 6B and 6C would have been indistinguishable, and troubleshooting would have been more complicated.

FIG. 7 illustrates a block diagram of an arrangement of a plurality of ported and process BA tracking barcodes 202, 204 with mixed DNA sample 706 used in a plurality of processes for subsequent identification of the two entities that provided first type of DNA sample molecule 702 and second type of DNA sample molecule 704, wherein process and ported NS tracking barcodes 202, 204 can be used to evaluate separately each of the process steps used to identify the two entities according to an embodiment. FIG. 7 illustrates DNA process 700 that uses both ported and process NS tracking barcodes 202, 204 according to aspects of the embodiments.

As shown in FIG. 7, both ported and process NS tracking barcodes 202, 204 can be used in a multi-step process to track different aspects of the process. In the DNA process flow of FIG. 7, a first type of DNA sample molecule 702 and a second type of DNA sample molecule 704 make up mixed DNA sample 706. When first type of DNA sample molecule 702 and second type of DNA sample molecule 704 (which make up mixed DNA sample 706) are combined with process NS tracking barcodes 204, the result is DNA process mixture 708. Following the creation of DNA process mixture 708 in a first step, DNA extraction occurs, which results in DNA process mixture 708′. Extraction is followed by amplification (708″), and then indexing. In the indexing step, ported NS tracking barcode 202 is added to DNA process mixture 708″ to create DNA process mixture 708′″. Finally, sequencing occurs in a next-to-final step (the final step can be analysis), wherein the mixture is now referred to as DNA process mixture 708″″. According to aspects of the embodiments, process NS tracking barcodes 204 were added in the extraction step to assess DNA extraction yield and to evaluate the yield of amplification (in the amplification step). Then, ported NS tracking barcodes 202 were added in the indexing step to aid in multiplexing that helps discriminate first type of DNA sample molecule 702 from second type of DNA sample molecule 704 if they are going to be sequenced in one pool.

As those of skill in the art can appreciate, many if not most of the steps and processes described herein can be performed by complicated but well-designed automated machinery, which allows skill technicians and/or highly educated professionals to perform the steps described herein, and evaluate the findings produced therefrom.

As those of skill in the art can further appreciate, such systems are generally automated, meaning that each can be controlled by one or more internally used computers, or microprocessors, and as such, each is therefore capable of being controlled as part of a larger network that can automate, to some degree or another, the entire or almost entire process. Such substantially or fully complete automation can include most if not all of the steps of method 100, as well as distribution of the data resulting from the analysis performed as a result of the findings. Because such systems are known to those of skill in the art, a detailed discussion thereof has been omitted in fulfillment of the dual purposes of clarity and precision.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and can include other examples that occur to those skilled in the art. Such other examples are intended to be within the scope of the claims.

The above-described embodiments are intended to be illustrative in all respects, rather than restrictive, of the embodiments. Thus the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

1-3. (canceled)

4. A nucleic acid sequence for use in a reaction, comprising:
a tracking barcode subunit made up of a first sequence of nucleic acids, the tracking barcode subunit being non-modifiable during the reaction; and
a process barcode subunit made up of a second sequence of nucleic acids, the process barcode subunit being modifiable during the reaction.

5. The nucleic acid sequence for use in a reaction according to claim 4, further comprising a subunit made up of endogenous nucleotides.

6. The nucleic acid sequence for use in a reaction according to claim 4, further comprising a subunit made up of endogenous nucleotides, wherein at least two subunits are contiguous.

7. The nucleic acid sequence for use in a reaction according to claim 4, further comprising a subunit made up of endogenous DNA.

8. The nucleic acid sequence for use in a reaction according to claim 4, further comprising a subunit made up of endogenous DNA, wherein at least two subunits are contiguous.

9. The nucleic acid sequence for use in a reaction according to claim 4, further comprising a subunit made up of endogenous RNA.

10. The nucleic acid sequence for use in a reaction according to claim 4, further comprising a subunit made up of endogenous RNA, wherein at least two subunits are contiguous.

11. The nucleic acid sequence for use in a reaction according to claim 1, wherein a Hamming distance between any two or more of said subunits being continguous in relation to one another is greater than one.

12. A method of tracking a reaction comprising,
using a nucleic acid sequence made up of any one of: (a) a ported barcode wherein the ported barcode does not undergo a structural modification in the reaction; and (b) a process barcode wherein the process barcode undergoes a structural modification in the reaction; and
determining one or both of: (c) a quantitative characteristic of the reaction, and (d) a qualitative characteristic of the reaction.

13. The method according to claim 12, wherein the quantitative characteristic of the reaction is determined using the ported barcode.

14. The method according to claim 12, wherein the qualitative characteristic of the reaction is determined using process barcode.

15. The method according to claim 12, wherein the nucleic acid sequence further comprises a sequence of endogenous nucleotides.

16. The method according to claim 12, wherein the nucleic acid sequence further comprises a sequence of endogenous DNA.

17. The method according to claim 12, wherein the nucleic acid sequence further comprises a sequence of endogenous RNA.

18. The method according to claim 12, wherein the efficiency of the reaction is determined by quantifying the amount of process barcode that is modified by the reaction to calculate the efficiency of the reaction.

19. The method according to claim 12, further comprising additional reactions and determining the efficiency of one or more of the additional reactions by molecular counting techniques to quantify the amount of modified process barcodes and unmodified ported barcodes to calculate conversion efficiency of one or more of the plurality of reactions.

20. The method according to claim 12, wherein a Hamming distance between any two or more of said subunits being continguous in relation to one another is greater than one.

21. A method of tracking a plurality of reactions comprising:
using a nucleic acid sequence made up: (a) a ported barcode wherein the ported barcode does not undergo a structural modification in any one of the plurality of reactions; and (b) a plurality of process barcodes wherein at least one of the process barcodes are selected to undergo a structural modification in one or more of the plurality of reactions; and
determining one or both of: (c) a quantitative characteristic of one or more of the plurality of reactions, and (d) a qualitative characteristic of one or more of the plurality of reactions.

22. The method according to claim 21, wherein the quantitative characteristic of one or more of the plurality of reactions is determined using the ported barcode.

23. The method according to claim 21, wherein the qualitative characteristic of one or more of the plurality of reactions is determined using one or more of the process barcodes.


SEQUENCE LISTING (15 sequences)

SEQ ID NO: 1 - DNA, 30 bp, Artificial Sequence
  Description of Artificial Sequence Synthetic oligonucleotide
actggcgtcctaccgtaggcttgaacgctg

SEQ ID NO: 2 - DNA, 12 bp, Artificial Sequence
  Description of Artificial Sequence Synthetic oligonucleotide
actggcacgctg

SEQ ID NO: 3 - DNA, 12 bp, Artificial Sequence
  Description of Artificial Sequence Synthetic oligonucleotide
ctgatcacgctg

SEQ ID NO: 4 - DNA, 18 bp, Artificial Sequence
  Description of Artificial Sequence Synthetic oligonucleotide
actggcacggaattctca

SEQ ID NO: 5 - DNA, 18 bp, Artificial Sequence
  Description of Artificial Sequence Synthetic oligonucleotide
gctccaacggaattctca

SEQ ID NO: 6 - DNA, 10 bp, Artificial Sequence
  Description of Artificial Sequence Synthetic oligonucleotide
gctccaacgg

SEQ ID NO: 7 - DNA, 18 bp, Artificial Sequence
  Description of Artificial Sequence Synthetic oligonucleotide
ctgatcacggaattctca

SEQ ID NO: 8 - DNA, 10 bp, Artificial Sequence
  Description of Artificial Sequence Synthetic oligonucleotide
ctgatcacgg

SEQ ID NO: 9 - DNA, 18 bp, Artificial Sequence
  Description of Artificial Sequence Synthetic oligonucleotide
actggcacgtcgctttac

SEQ ID NO: 10 - DNA, 18 bp, Artificial Sequence
  Description of Artificial Sequence Synthetic oligonucleotide
gctccaacgtcgctttac

SEQ ID NO: 11 - DNA, 18 bp, Artificial Sequence
  Description of Artificial Sequence Synthetic oligonucleotide
ctgatcacgtcgctttac

SEQ ID NO: 12 - DNA, 18 bp, Artificial Sequence
  Description of Artificial Sequence Synthetic oligonucleotide
autgguaugtugctttau

SEQ ID NO: 13 - DNA, 18 bp, Artificial Sequence
  Description of Artificial Sequence Synthetic oligonucleotide
gutuuaaugtugutttau

SEQ ID NO: 14 - DNA, 18 bp, Artificial Sequence
  Description of Artificial Sequence Synthetic oligonucleotide
utgatuaugtugutttau

SEQ ID NO: 15 - DNA, 12 bp, Artificial Sequence
  Description of Artificial Sequence Synthetic oligonucleotide
ggtgctaggctc